(19) United States
(12) Patent Application Publication
(10) Pub. No.: US 2023/0404746 A1
(43) Pub. Date: Dec. 21, 2023

(54) LENGTH-ADJUSTABLE OSSICULAR PROSTHESIS HAVING IN-SITU ELONGATION OUT OF THE HEAD PLATE

(71) Applicant: Heinz Kurz GmbH, Dusslingen (DE)

(72) Inventors: Thomas STIEGELE, Riederich (DE); Alexander SANDER, Titisee-Neustadt (DE); Matthias MERTENS, Kaltenkirchen (DE)

(73) Assignee: Heinz Kurz GmbH, Dusslingen (DE)

(21) Appl. No.: 18/208,949

(22) Filed: Jun. 13, 2023

(30) Foreign Application Priority Data
Jun. 15, 2022 (DE) 20 2022 103 367.6

(51) Int. Cl. A61F 2/18

(52) U.S. Cl. CPC A61F 2/18; A61F 2002/183; A61F 2250/0007; A61F 2230/0093; A61F 2220/0008; A61F 2210/0047

(57) ABSTRACT

An ossicular prosthesis has a first fastening element for mechanical contact with the tympanic membrane, and a second fastening element for connecting to a member of the ossicular chain, and a connecting element which connects the fastening elements and which has rib elements which can be spread radially outwards and thereby shorten the axial length of the ossicular prosthesis. The rib elements lead directly into coupling regions of the first fastening element and into a coupling element which is rigidly connected to the second fastening element. When a force is introduced in parallel with the longitudinal axis in the direction, the rib elements assume a position located radially further from the longitudinal axis, and shorten the functional length of the prostheses, or increase with the introduction of an antiparallel force.

### CROSS REFERENCE TO RELATED APPLICATIONS

Applicant claims priority under 35 U.S.C. § 119 of German Application No. 20 2022 103 367.6 filed Jun. 15, 2022, the disclosure of which is incorporated by reference.

### BACKGROUND OF THE INVENTION

### 1. Field of the Invention

The invention relates to an ossicular prosthesis which replaces or bridges at least one member or parts of a member of the ossicular chain, the ossicular prosthesis comprising, at one end thereof, a first fastening element designed as a head plate for mechanical contact with the tympanic membrane and/or with the handle of malleus, and, at its other end, a second fastening element for mechanical connection to a member or parts of a member of the ossicular chain or to the inner ear, and a connecting element which connects the two fastening elements to one another in a sound-conducting manner along a longitudinal axis, and the connecting element having rib elements which, at least sectionally, can be spread to a greater or lesser extent, radially outwards away from the longitudinal axis, and thereby shorten the axial length of the ossicular prosthesis to a greater or lesser extent.

### 2. Description of the Related Art

Such a device is known from U.S. Pat. No. 10,687,937 B2 (=reference [1]).

Ossicular prostheses are used to transfer the sound from the tympanic membrane to the inner ear in the case of completely or partially missing or damaged ossicles of the human middle ear. In this case, the ossicular prosthesis has two ends, depending on the specific circumstances one end of the ossicular prosthesis resting, for example by means of a head plate, on the tympanic membrane or the handle of malleus, and the other end of the ossicular prosthesis being fastened, for example, to the stirrup bone of the human ossicular chain or being dipped directly into the inner ear. In many cases, the known ossicular prostheses allow sound transmission between the tympanic membrane and the inner ear only to a limited extent because they can only replace the natural anatomical formations of the ossicular chain in a very limited manner. (See, for example, DE 42 10 235 C1; EP 0 809 982 B1; U.S. Pat. No. 6,387,128 B1 (=reference [2])).

After the prosthesis has been surgically placed in the middle ear and the tympanic membrane is closed again, the so-called healing phase begins. During this time, scars are formed, and they cause unpredictable forces which may result in the prosthesis moving from its local position. In the case of a rigid connection between the head plate and the shaft, increased pressure peaks can occur between the edge of the head plate and the tympanic membrane or the graft between the tympanic membrane and the head plate. These can be so high that penetration or extrusion through the tympanic membrane may result. For this reason, it is very helpful if the prosthesis has a certain post-operative mobility so that the head plate can automatically adapt to the position of the tympanic membrane post-operatively.

Moreover, since the anatomical conditions of the ear, such as the position, the shape and the size of the stirrup bone, the anvil, the hammer and the tympanic membrane vary, it is very advantageous if ossicular prostheses are not designed to be rigid but have a certain flexibility or variability.

In order to achieve such flexibility/variability, various fastening and coupling devices for ossicles, which have resilient parts and/or joints, are known. Such an articulated connection between a fastening element, mountable on the stirrup foot plate, and the elongate shaft is described for example in EP 1 181 907 B1 (=reference [3]) and is offered by the applicant under the brand name “Ball Joint”.

A further complication, which occurs in various ways, arises due to under ventilation of the middle ear space and associated inflammation, tumor formations, adhesive processes in the tympanic membrane region and stiffening of the latter. For example, in the case of a dysfunction of the Eustachian tube, a negative pressure can occur in the middle ear, which can cause a bulge or protrusion (known as retraction) of the tympanic membrane and consequently adhesion for example to the stirrup bone. In order to counteract this and to be able to follow postoperative movements of the tympanic membrane, in known ossicular prostheses, the head plates are designed to be tiltable relative to the connecting element which connects the head plate to the second fastening element and is designed in most cases as an elongate shaft. Such a head plate which is inherently rigid but tiltable relative to the connecting element is described, inter alia, in US 2004/0162614 A1 (=reference [4]).

While reference [3] has an ossicular prosthesis in which the head plate has a built-in ball joint connected to the connecting shaft of the two fastening elements, EP 1 833 424 B1 (=reference [5]) even discloses a connecting shaft designed as a ball chain. As a result, the final axial length of the prosthesis can be set, at least in a stepped manner, by the selection of a certain number of balls in the chain and cutting off the excess balls.

However, a disadvantage of these known ossicular prostheses is that, due to the rigid tilting of the head plate during local medial movements of the tympanic membrane, the opposite side of the head plate is simultaneously moved laterally outwards, whereby pressure peaks are generated on the tympanic membrane.

EP 1 972 307 B1 (=reference [6]) discloses an ossicular prosthesis with a head plate designed to be highly flexible in which both the advantages of the prosthesis according to the above-mentioned reference [5] and also the advantages of the prostheses described in reference [4] are maintained, but the common disadvantages of a rigid tilting of the head plate are prevented.

A problem with the ossicular prosthesis according to reference [6], however, is its limited insertability into the middle ear of the patient, because, in particular in the radial direction with respect to its longitudinal axis, the head plate projects significantly laterally and can only be surgically inserted into the middle ear through the tympanic membrane region, by means of a large artificial opening. This large opening then of course also grows closed post-operatively with difficulty, and also leaves correspondingly large scars.

The head plate comprises, in reference [6], flexible rib elements which run within the head plate plane and connect a radially outer annular region of the head plate to a central coupling region arranged radially in the center of the head plate. These rib elements, together with the radially outer annular region and the central coupling region, are a fixed component of the head plate disclosed in the reference [6]. They are geometrically designed in such a way that they follow such a medial movement—also locally—during local medial movements of the tympanic membrane, but do not transmit the movement to remote regions of the head plate.

However, the rib elements are not designed for any radial compression of the head plate within the head plate plane and therefore a radial reduction of the head plate diameter, possibly for the purpose of facilitating passage through the tympanic membrane, and would be unsuitable for such a use. This is in particular because the radially outer annular region is rigid and therefore does not follow the attempt of such radial compression or would at most bend uncontrollably.

The ossicular prosthesis described in reference [6] is also entirely unsuitable for a change of length in the direction of the z-axis because, although the connecting element, which extends along the z-axis and rigidly connects the head plate to the second fastening element, is designed to transmit sound, it is designed as such to be rigid. Flexibility of the length of the connecting element in the z-direction is excluded and also not desired at all in the ossicular prosthesis according to reference [6].

EP 3 311 773 B1 (=reference [7]), in contrast, describes an ossicular prosthesis with a head plate that can be folded like an umbrella, and by means of which the prosthesis can be inserted into the middle ear in a virtually minimally invasive manner through a small opening in the tympanic membrane.

Another important issue in the implantation of ossicular prostheses is the setting of the correct axial length of the prosthesis that is optimally adapted to the individual circumstances and geometric conditions in the middle ear of the patient.

Reference [5], discussed above, already proposes a ball chain having separable end balls for this purpose. However, this unfortunately does not allow continuous, but merely a stepwise, length adjustment.

A length-variable ossicular prosthesis having a displacement mechanism, which is installed in the connecting element between the first and second fastening elements, for stepless length adjustment is described in DE 10 2007 041 539 B4 (=reference [8]).

Instead of such a displacement mechanism, which is relatively complex to produce, EP 2 238 946 B1 (=reference [9]) proposes a length-variable ossicular prosthesis in which an accordion-like structure is installed in the connecting element. The axial length of the ossicular prosthesis can then be shortened by an axial compression of this structure and increased by pulling it out.

EP 2 601 909 B1 (=reference [10]) in turn provides a displacement mechanism in the connecting element for the axial length adjustment of the ossicular prosthesis, which displacement mechanism comprises a receiving part and an insertion part which surrounds the receiving part with two legs in a clamp-like manner, the receiving part and the insertion part being displaceable relative to one another in the axial direction of the connecting element.

Finally, a laser-activatable length-variable ossicular prosthesis is disclosed in EP 3 130 315 B1 (=reference [11]). It is proposed therein to construct the connecting element having stretchable and/or compressible partial strands, folded in a loop-like manner, which are made of a shape-memory material. Activation surfaces are connected to the loops of these partial strands in a heat-conducting manner, which surfaces can cause thermal activation, and therefore a deformation of the loops, by the action of heat. In this way, the axial length of the ossicular prosthesis can be changed and adjusted as desired.

Reference [1], already cited above, finally discloses a generic ossicular prosthesis having all of the sets of feature defined at the outset. In particular, it is proposed to provide strip-like rib elements in the connecting element, which rib elements can be spread radially outwards away from the longitudinal axis and in the process shorten the axial length of the connecting element and therefore the axial length of the ossicular prosthesis. The rib elements are spread from the side, i.e. in the radial direction with respect to the connecting element. However, an increase in the axial length is not provided in this mechanism of action. Without additional measures, this length adjustment is also possible only before the prosthesis is inserted into the middle ear of the patient, but not in situ in the implanted state.

### SUMMARY OF THE INVENTION

In contrast, the object of the present invention is to improve a generic length-adjustable ossicular prosthesis of the type defined at the outset using the simplest possible technical means in such a way that a length adjustment in situ of an already placed prosthesis can take place in an uncomplicated and cost-effective manner, that the length adjustment of the prosthesis is also possible from above, that a uniform and symmetrical elongation of the prosthesis is ensured in the process, and that a use of the invention is also made possible, for example, with partial prostheses having a small axial overall height and/or a small functional length.

According to the invention, this object is achieved in a manner that is as surprisingly simple as it is effective in that the rib elements lead at one end directly into coupling regions of the first fastening element within the head plate plane and are movably, but non-detachably, connected thereto, in that all the rib elements lead directly, at the other end, into a coupling element and are also movably, but non-detachably, connected thereto, the coupling element in turn being rigidly connected at the other end to the second fastening element, in that the rib elements are designed such that, in an in-situ state of the ossicular prosthesis inserted in a human middle ear, when a force is applied to the rib elements with the force component in parallel with the longitudinal axis in the direction from the first fastening element to the second fastening element, they in each case sectionally assume, in situ, a position located radially further from the longitudinal axis, and therefore shorten, in situ, the axial functional length between the first fastening element and the second fastening element, the rib elements, with the introduction of a force having force components that are antiparallel with respect to the longitudinal axis, in the direction from the second fastening element to the first fastening element, in each case sectionally assuming, in situ, a position located radially closer to the longitudinal axis, and therefore increasing, in situ, the axial functional length between the first fastening element and the second fastening element, and in that the rib elements retain, in situ, their adjusted radial position with respect to the longitudinal axis, when no force acts.

As a result, the advantages of the above-described generic ossicular prosthesis, as described in reference [1], can be used relatively simply, it being possible, however, for the change in length to take place when the prosthesis is already placed within the middle ear, which would not be possible or would be possible only with very significant changes with the prosthesis in reference [1].

Due to the possibility of converting a mechanical movement within the surface of the head plate into an axial length adjustment, the axial movement ultimately always leading out of the head plate plane, with the ossicular prosthesis according to the invention, a finely adjustable length setting can also be carried out only after insertion into the middle ear, i.e. in situ. In this case, the change in length, that is to say the change in the distance between the head plate and the second fastening element at the other end of the prosthesis, is achieved by expansion from the head plate plane downwards (or vice versa).

The mechanism according to the invention is automatically guided within the head plate and therefore ensures uniform and above all symmetrical elongation even without additional measures. In contrast to this, the approaches of the prior art, in particular in reference [1], describe solutions that require the user to manipulate the prosthesis on both sides in order to bring about a symmetrical change in length.

Since the mechanism for length adjustability is integrated in the head plate and is installed with it, the ossicular prosthesis according to the invention can also be produced with a very low overall height.

In addition, the mechanism according to the invention can be operated from above in the head plate. This is also a significant difference from the prior art. For example, the prostheses according to reference [1] enable only a cumbersome manipulation from the side which requires a relatively large amount of space. In contrast, the approach according to the invention has an extremely space-saving design and is also suitable for endoscopic accesses.

Particularly preferred embodiments of the ossicular prosthesis according to the invention are characterized in that the rib elements are designed such that their adjusted radial position relative to the longitudinal axis can be reversibly changed in situ by introducing a corresponding force.

In particular, the movement of the prosthesis parts during the adjustment process can therefore be made reversible, and the prosthesis length can therefore not only be increased, but also shortened again.

The low introduction of force ensures that surrounding middle ear structures are not damaged.

The sophisticated design, according to the invention, of the head plate allows optimum visibility conditions during the surgery.

By means of the lightweight construction, optimal sound transmission is made possible, in particular in the high-frequency range.

As a result of the head plate design according to the invention, optional coupling to the malleus manubrium was made possible.

The design according to the invention was developed such that there are no dead spaces. Therefore, no biofilms can form on the implant and develop implant-associated infections.

Further advantageous embodiments of the invention are characterized in that locking devices are present which, when an axial force is applied to the rib elements, each cause a mechanical resistance at one or more axial lengths of the connecting element.

The adjustment mechanism is therefore provided with locking points in order to achieve a defined axial length of the prosthesis and to fix this length.

The low force application ensures that surrounding middle ear structures are not damaged.

The locking devices enable optimal visibility conditions during the surgery.

The implant can be picked up again during the surgery to ensure a readjustment.

Embodiments are also preferred in which the coupling regions arranged within the head plate plane of the first fastening element are geometrically designed such that they can be used to apply a force to the rib elements with the force component in parallel or antiparallel with respect to the longitudinal axis in situ by means of an adjusting tool.

The in-situ length adjustment of the ossicular prosthesis can be achieved by manipulating the structure within the head plate. If the structure is pressed together using an instrument, the prosthesis is extended. If the structure is spread, the length is shortened. In this case, the adjusting tool serves as an implantation aid, and in particular for operating the mechanism for adjusting the length. It can be designed as a minimally invasive, in particular endoscopic, instrument, preferably tweezer-like or pincer-like.

A change in length of the ossicular prosthesis can in principle take place by plastic deformation of the connecting struts and/or by integrated joints.

Therefore, embodiments of the invention are advantageous in which the connection points of the rib elements to the coupling regions of the first fastening element, and the connection points of the rib elements to the coupling element, are each designed as mechanical joints or bending points.

A first class of embodiments of the ossicular prosthesis according to the invention is characterized in that the rib elements are designed so as to be mechanically rigid at least in part, preferably completely.

In advantageous developments of this first class of embodiments, integrated mechanical joints or bending points are formed between the mechanically rigid portions of the rib elements.

In this way, a certain flexibility or variability of the prosthesis can be achieved, as is described per se in reference [3]. In view of a particularly high post-operative mobility of the prosthesis, developments can be used in which a plurality of adjacent further rotary elements, preferably a ball joint chain, are used.

In a second class of alternative embodiments of the invention, the rib elements are made of a plastic, flexible material, at least sectionally, the plastic, flexible material of the rib elements having, in particular, an elasticity of 1%, preferably an elasticity of 2%.

If the rib elements consist of flexible material, the space and the technical effort for the mechanical pivot joints can be saved. If the material of the rib elements has flexibility of >1%, the prosthesis can be constructed particularly “tightly”, it also being ensured that the prosthesis can follow the smallest “topographical” changes in the tympanic membrane.

In a first group of developments of this second class of embodiments, the plastic, flexible material of the rib elements contains highly elastic material, preferably amorphous metal, in particular based on nickel, iron, cobalt or zirconium, and/or a nickel-titanium alloy and/or memory metal.

The above-mentioned properties of flexibility together with good stiffness for sound conduction can be optimally achieved using the mentioned materials.

In particular, the material titanium has, in addition to its strength and excellent sound transmission properties, also an excellent biocompatibility with the human middle ear as is known. With regard to post-operative position adjustment, embodiments of the invention are advantageous in which the prosthesis or parts thereof, in particular one of the fastening elements, are made of a material having memory effect or superelastic properties, preferably of nitinol, which is known per se for example from EP 1 961 400 B1 (=reference [12]).

In a second group of developments, however, the plastic, flexible material of the rib elements can also contain highly elastic plastic, in particular high-strength elastic polymer, and/or elastic ceramic.

These materials enable ideal preconditions for good sound transmission, with high flexibility and good rigidity, but lower density.

Developments of the above-described second class of embodiments are particularly advantageous in which the coupling regions, the rib elements and the coupling element are arranged flat within the head plate plane of the first fastening element before the first application of a force to the rib elements with a force component in parallel or antiparallel with respect to the longitudinal axis.

This enables a particularly compact construction of the ossicular prosthesis according to the invention.

Preferred variants of these developments are characterized in that the rib elements extend in a curved and/or meandering and/or zigzag manner between their respective coupling region and the coupling element.

Due to a profile which is not linear but curved instead, the desired effect increases of a local flexibility of the head plate and an only locally limited deviation from smaller medial movements of the tympanic membrane. In addition, the head plate can therefore more easily follow any post-operative change in the tympanic membrane.

In addition, these developments can also be improved in that the coupling element is constructed in an annular shape and is preferably arranged centrally in the first fastening element in its initial position within the head plate plane.

This measure also enables a particularly compact construction of the ossicular prosthesis according to the invention. The mechanism is “pressed out” from the head plate by means of a central attachment point. The axial length of the prosthesis expands by plastic deformation.

In embodiments of the invention, radially outer, free end portions of the rib elements can have atraumatic, non-pointed, free end edges which are radially to the outside with respect to the longitudinal axis and are expanded transversely to the direction of the longitudinal axis, or can be designed as atraumatic surfaces, preferably as closed surfaces or annular surfaces, in particular circular or elliptical. Thus, if a tilting torque arises in the healing phase which results in an increased force effect on the outer end portions, the surface pressure and the risk of extrusion can be reduced.

In further embodiments of the invention, the second fastening element is designed as a plate, in particular as a curved plate, as a sleeve, as a loop, as a closed bell, in particular in the form of a hollow cylinder, as a singly or multiply slotted bell, or as a clip for mechanical connection to a further member of the ossicular chain. Alternative embodiments can provide for the ossicular prosthesis to be coupled directly to the inner ear, at its end supporting the second fastening element, by perforating the stirrup foot plate (=stapedectomy or stapedotomy) and/or by opening the human cochlea (=cochleotomy), in particular by means of a piston.

In addition to post-operative position displacement, a further problem also arises after the implantation of ossicular prostheses: Specifically, the middle ear of the human body constitutes a “half-open bearing”. Each implantation material which is introduced into the body in the context of a reconstruction of the middle ear and its structures, thereby experiences a particular stress due to the fact that a contaminated and infected environment prevails, which usually attacks the material. Since the aim of the implantation of an ossicular prosthesis must also always be to keep the implant in the middle ear of the patient for as long as possible without complications, a prolonged attack on the material can lead to damage to the prosthesis and/or to local infection. Both consequences are not tolerable. In order to permanently prevent damage to both the implantation material and the surrounding tissue, in a further particularly preferred embodiment of the invention, the surface of the ossicular prosthesis is completely or at least partially coated with a biologically active coating, in particular a growth-inhibiting and/or a growth-promoting and/or an antibacterial coating. The head plate of the ossicular prosthesis according to the invention should in principle have a growth-promoting coating, while a second fastening element, which leads directly into the inner ear and is designed for example in the form of a piston, has a growth-inhibiting coating.

Alternatively or additionally, in further embodiments, parts of the ossicular prosthesis according to the invention can be made from a ceramic material. However, embodiments of the invention are also possible in which the prosthesis or parts thereof are made of biocompatible plastics, in particular silicone, polytetrafluoroethylene (PTFE) or fiber composite materials.

These materials can also prevent post-operative rejection reactions in most cases.

A system comprising an ossicular prosthesis constructed according to the invention of the type described above, and an adjusting tool which can also serve as an implantation aid and which can also be designed as a minimally invasive, in particular endoscopic, instrument, preferably tweezer-like or pincer-like, is also covered by the scope of the present invention.

Less trauma in the surgical implantation of the ossicular prosthesis is advantageous here, since only a smaller opening of the tympanic membrane, etc. is necessary.

Finally, a method for implantation of an ossicular prosthesis according to the invention of the type described above, which is characterized in that the first fastening element, in particular the coupling regions within the head plate plane, has structures made of memory metal, and in that a force is applied to the rib elements by heating the structures made of memory metal, preferably in a contactless manner, in particular by light irradiation, for example laser radiation, and the resulting deformation of said memory metal, is also covered by the scope of the present invention.

In particular, contactless force application into the ossicular prosthesis according to the invention, for example by means of thermal radiation, is therefore made possible.

### BRIEF DESCRIPTION OF THE DRAWINGS

Other objects and features of the invention will become apparent from the following detailed description considered in connection with the accompanying drawings. It is to be understood, however, that the drawings are designed as an illustration only and not as a definition of the limits of the invention.

In the drawings,

FIG. 1A) to FIG. 1D) show four illustrations of a schematic three-dimensional view of a first embodiment of the ossicular prosthesis according to the invention, of which three illustrations are in an oblique view from above with viewing direction from the second to the first fastening element, the rib elements being extended:

FIG. 1A) only very little,

FIG. 1B) a moderate amount,

FIG. 1C) virtually completely in the z-direction from the head plate plane, and a fourth illustration:

FIG. 1D) showing a lateral view of the ossicular prosthesis in a viewing direction slightly obliquely from below onto the head plate;

FIG. 2A) to FIG. 2C) shows a further embodiment of a head plate designed according to the invention, namely:

FIG. 2A) with a slightly oblique view towards the ossicular prosthesis in the viewing direction from the second to the first fastening element, the rib elements still being arranged completely in the head plate plane before an axial force is introduced,

FIG. 2B) like FIG. 2A) but with slightly extended rib elements,

and

FIG. 2C) the head plate belonging to this embodiment, viewed in the z direction from above;

FIG. 3 shows a further embodiment of an ossicular prosthesis designed according to the invention, in which the connecting element between the first and second fastening elements comprises two parallel rigid shaft pieces; and

FIG. 4 shows an embodiment of an ossicular prosthesis according to the invention in which the first fastening element is designed as a divided head plate in two parallel planes perpendicular to the z-axis, the connecting element between the first and second fastening elements in each case comprising a shaft piece running in each of the two head plate planes.

### DETAILED DESCRIPTION OF PREFERRED EMBODIMENTS

The embodiments of the ossicular prosthesis 10; 20; 30; 40 (or parts thereof) according to the invention, shown schematically in the figures of the drawing, each comprise a first fastening element 11; 21; 31; 41 at one end, which is designed in the form of a flat head plate for mechanical contact with the tympanic membrane and/or with the handle of malleus. At the other end of the ossicular prosthesis 10; 20; 30; 40 sits a second fastening element 12; 22; 32; 42 for mechanically connecting the prosthesis to a member or parts of a member of the ossicular chain or to the inner ear. A connecting element 13 which connects the two fastening elements 11, 12 or 21, 22 or 31, 32 or 41 to one another in a sound-conducting manner along a longitudinal axis z, and which in some embodiments can be designed in the form of a—usually elongate—shank, is arranged therebetween.

The connecting element 13 comprises rib elements 14; 24; 34; 44 which, at least sectionally, can be spread, to a greater or lesser extent, radially outwards from the longitudinal axis z, and thereby shorten the axial length of the ossicular prosthesis 10; 20; 30; 40 to a greater or lesser extent.

In comparison to known generic ossicular prostheses, the invention is characterized in that the rib elements 14; 24; 34; 44 lead at one end directly into coupling regions 15; 25; 35; 45 of the first fastening element 11; 21; 31; 41 within the head plate plane and are movably, but non-detachably, connected thereto,
in that all the rib elements 14; 24; 34; 44 lead directly, at the other end, into a coupling element 16; 26; 36; 46 and are also movably, but non-detachably, connected thereto, the coupling element 16; 26; 36; 46 in turn being rigidly connected at the other end to the second fastening element 12; 22; 32; 42,
in that the rib elements 14; 24; 34; 44, in an in-situ state of the ossicular prosthesis 10; 20; 30; 40 inserted in a human middle ear, when a force is applied to the rib elements 14; 24; 34; 44 with the force component in parallel with the longitudinal axis z in the direction from the first fastening element 11; 21; 31; 41 to the second fastening element 12; 22; 32; 42, in each case sectionally assume, in situ, a position located radially further from the longitudinal axis z, and therefore shorten, in situ, the axial functional length between the first fastening element 11; 21; 31; 41 and the second fastening element 12; 22; 32; 42, the rib elements 14; 24; 34; 44, with the introduction of a force having force components that are antiparallel with respect to the longitudinal axis z, in the direction from the second fastening element 12; 22; 32; 42 to the first fastening element 11; 21; 31; 41, in each case sectionally assuming, in situ, a position located radially closer to the longitudinal axis z, and therefore increasing, in situ, the axial functional length between the first fastening element 11; 21; 31; 41 and the second fastening element 12; 22; 32; 42,
and in that the rib elements 14, 24; 34; 44 retain, in situ, their adjusted radial position with respect to the longitudinal axis z, when no force acts.

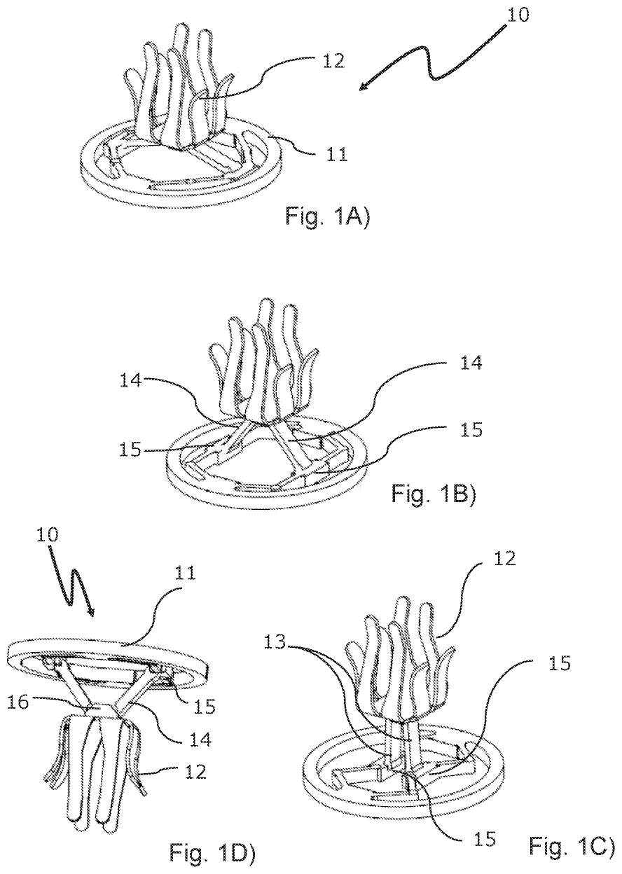

The three views FIG. 1A) to FIG. 1C) show the ossicular prosthesis 10 in three different operating positions, namely:
FIG. 1A) in an only very slightly extended state of the rib elements 14, in which said rib elements are still arranged virtually in the head plate plane of the first fastening element 11,
FIG. 1B) having a moderate extension path of the rib elements 14 and correspondingly enlarged functional length of the prosthesis between the first fastening element 11 and the second fastening element 12, and
FIG. 1C) having rib elements 14 extending fully in the z-direction, out of the head plate plane.

The fourth view FIG. 1D) shows the ossicular prosthesis 10 laterally obliquely from below, with a viewing direction from the second fastening element 12 to the first fastening element 11, in a “semi-extended” state of the rib elements 14 (such as in FIG. 1B)). Here, the coupling element 16 can be clearly seen.

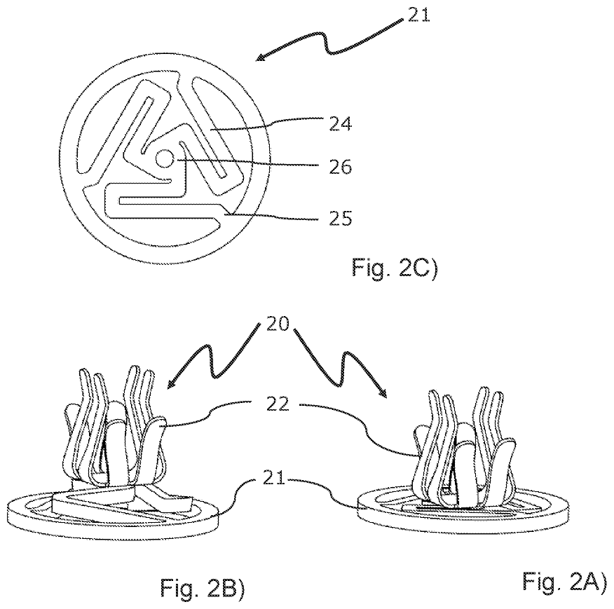

The two first views FIG. 2A) and FIG. 2C) show two operating positions of a further embodiment of the ossicular prosthesis 20 according to the invention, namely:
FIG. 2A) having rib elements 24 still arranged completely in the head plate plane prior to the introduction of an axial force, and
FIG. 2B) having helically designed rib elements extended somewhat in the z-direction, after an axial force has been introduced.

The third view FIG. 2C) shows the head plate 21 belonging to this embodiment with a plan view from above in the z-direction.

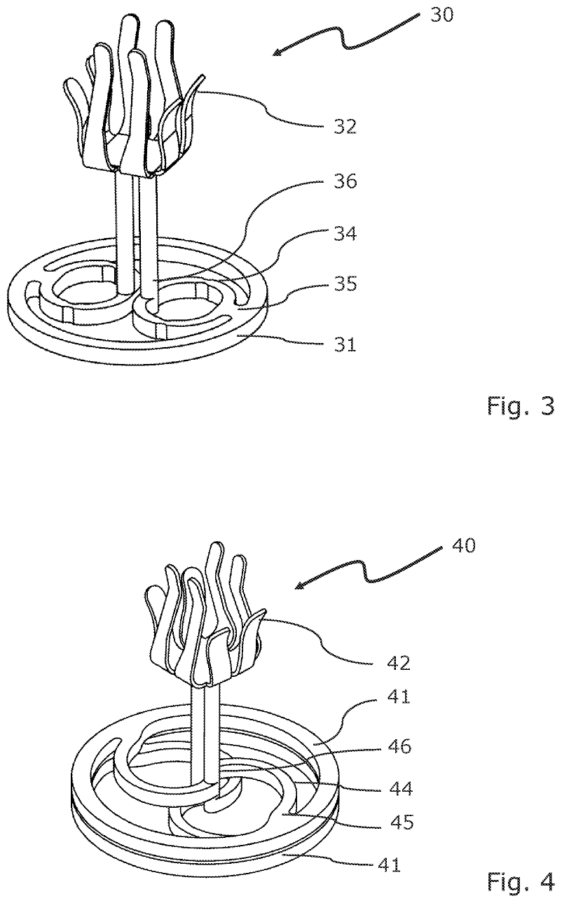

FIG. 3 shows a further embodiment of an ossicular prosthesis 30 designed according to the invention, in which the connecting element between the first fastening element 31 and the second fastening element 32 comprises two rigid shaft pieces extending in parallel with the z-axis. One end of these two shaft pieces is used here as a coupling element 36, into which the annular rib elements 34 lead at their end facing the second fastening element 32, proceeding from their respective coupling region 35 in the first fastening element 31.

FIG. 4 finally shows a further variant of an ossicular prosthesis 40 according to the invention, in which the first fastening element 41 is designed as a divided head plate in two parallel planes. The connecting element between the first fastening element 41 and the second fastening element 42 in turn comprises two parallel shaft pieces, the two shaft pieces having different lengths in the z-direction, however, due to the axial distance between the two halves of the first fastening element 41. Here too, the end of these two shaft pieces facing the first fastening element 41 serves in each case as a coupling element 46 for the rib elements 44, proceeding from their respective coupling region 45 in the first fastening element 41. In the second fastening element 42, both shaft pieces then end at the same axial height.

Although only a few embodiments of the present invention have been shown and described, it is to be understood that many changes and modifications may be made thereunto without departing from the spirit and scope of the invention.

### LIST OF REFERENCE SIGNS

10; 20; 30; 40 ossicular prosthesis
11; 21; 31; 41 first fastening element
12; 22; 32; 42 second fastening element
13 connecting element
14; 24; 34; 44 rib elements
15; 25; 35; 45 coupling regions
16; 26; 36; 46 coupling element
Z longitudinal axis

### LIST OF REFERENCES

Publications considered for the assessment of patentability:
[1] U.S. Pat. No. 10,687,937 B2
[2] DE 42 10 235 C1; EP 0 809 982 B1; U.S. Pat. No. 6,387,128 B1
[3] EP 1 181 907 B1
[4] US 2004/0162614 A1
[5] EP 1 833 424 B1
[6] EP 1 972 307 B1
[7] EP 3 311 773 B1
[8] DE 10 2007 041 539 B4
[9] EP 2 238 946 B1
[10] EP 2 601 909 B1
[11] EP 3 130 315 B1
[12] EP 1 961 400 B1

What is claimed is:

1. An ossicular prosthesis (10; 20; 30; 40), which replaces or bridges at least one member or parts of a member of an ossicular chain, the ossicular prosthesis (10; 20; 30; 40) comprising:
at one end, a first fastening element (11; 21; 31; 41) formed as a flat head plate configured for mechanical contact with a tympanic membrane and/or with a handle of malleus,
at the other end, a second fastening element (12; 22; 32; 42) configured for mechanical connection to a member or parts of a member of the ossicular chain or to the inner ear, and
a connecting element (13) that connects the two fastening elements (11, 12; 21, 22; 31, 32; 41, 42) to one another, in a sound-conducting manner, along a longitudinal axis (z), the connecting element (13) having rib elements (14; 24; 34; 44), which are configured to be spread outwards, to a greater or lesser extent, radially away from the longitudinal axis (z), at least sectionally, so as to shorten an axial length of the ossicular prosthesis (10; 20; 30; 40) to a greater or lesser extent,
wherein
the rib elements (14; 24; 34; 44) lead at the one end directly into coupling regions (15; 25; 35; 45) of the first fastening element (11; 21; 31; 41) within a head plate plane and are movably, but non-detachably, connected thereto,
all the rib elements (14; 24; 34; 44) lead directly, at the other end, into a coupling element (16; 26; 36; 46) and are also movably, but non-detachably, connected thereto, the coupling element (16; 26; 36; 46) in turn being rigidly connected at the other end to the second fastening element (12; 22; 32; 42),
the rib elements (14; 24; 34; 44) are designed in such a way that, in an in-situ state of the ossicular prosthesis (10; 20; 30; 40) inserted in a human middle ear, when a force is applied to the rib elements (14; 24; 34; 44) with a force component in parallel with the longitudinal axis (z) in a direction from the first fastening element (11; 21; 31; 41) to the second fastening element (12; 22; 32; 42) in each case sectionally assume, in situ, a position located radially further from the longitudinal axis (z), and therefore shorten, in situ, the axial functional length between the first fastening element (11; 21; 31; 41) and the second fastening element (12; 22; 32; 42), each of the rib elements (14; 24; 34; 44), with the introduction of a force having force components that are antiparallel with respect to the longitudinal axis (z), in the direction from the second fastening element (12; 22; 32; 42) to the first fastening element (11; 21; 31; 41) sectionally assuming, in situ, a position located radially closer to the longitudinal axis (z), and therefore increasing, in situ, the axial functional length between the first fastening element (11; 21; 31; 41) and the second fastening element (12; 22; 32; 42),
and the rib elements (14, 24; 34; 44) retain, in situ, their adjusted radial position with respect to the longitudinal axis (z), when no force acts.

2. The ossicular prosthesis according to claim 1, wherein the rib elements (14; 24; 34; 44) are designed such that their adjusted radial position relative to the longitudinal axis (z) can be reversibly changed in situ by introducing a corresponding force.

3. The ossicular prosthesis according to claim 1, further comprising locking devices which are configured to cause a mechanical resistance at one or more axial lengths of the connecting element (13) when an axial force is applied to the rib elements (14; 24; 34; 44).

4. The ossicular prosthesis according to claim 1, wherein the coupling regions (15; 25; 35; 45) arranged within the head plate plane of the first fastening element (11; 21; 31; 41) are geometrically designed such that they can be used to apply a force to the rib elements (14; 24; 34; 44) with a force component in parallel or antiparallel with respect to the longitudinal axis (z) by means of an adjusting tool in situ.

5. The ossicular prosthesis according to claim 1, wherein the connection points of the rib elements (14; 24; 34; 44) with the coupling regions (15; 25; 35; 45) of the first fastening element (11; 21; 31; 41) and the connection points of the rib elements (14; 24; 34; 44) with the coupling element (16; 26; 36; 46) are each designed as mechanical joints or bending points.

6. The ossicular prosthesis according to claim 1, wherein the rib elements (14; 24; 34; 44) are designed so as to be mechanically rigid at least sectionally.

7. The ossicular prosthesis according to claim 6, wherein integrated mechanical joints or bending points are formed between the mechanically rigid portions of the rib elements (14; 24; 34; 44).

8. The ossicular prosthesis according to claim 1, wherein the rib elements (14; 24; 34; 44) are formed at least sectionally from a plastic, flexible material, and wherein the plastic, flexible material of the rib elements (14; 24; 34; 44) has an elasticity of 1%, preferably an elasticity of 2%.

9. The ossicular prosthesis according to claim 8, wherein the plastic, flexible material of the rib elements (14; 24; 34; 44) contains highly elastic material in the form of amorphous metal, based on nickel, iron, cobalt or zirconium, and/or a nickel-titanium alloy and/or memory metal.

10. The ossicular prosthesis according to claim 8, wherein the plastic, flexible material of the rib elements (14; 24; 34; 44) contains a highly elastic plastic in the form of a high-strength elastic polymer, and/or elastic ceramic.

11. The ossicular prosthesis according to claim 8, wherein the coupling regions (15; 25; 35; 45), the rib elements (14; 24; 34; 44) and the coupling element (16; 26; 36; 46) are arranged flat within the head plate plane of the first fastening element (11; 21; 31; 41) before the first application of a force to the rib elements (14; 24; 34; 44) with a force component in parallel or antiparallel with respect to the longitudinal axis (z).

12. The ossicular prosthesis according to claim 11, wherein the rib elements (14; 24; 34; 44) extend between their respective coupling region (15; 25; 35; 45) and the coupling element (16; 26; 36; 46) in a curved and/or meandering and/or zigzag manner.

13. The ossicular prosthesis according to claim 11, wherein the coupling element (16; 26; 36; 46) has a ring-shaped design and is arranged centrally in the first fastening element (11; 21; 31; 41) in an initial position within the head plate plane.

14. A system comprising an ossicular prosthesis (10; 20; 30; 40) according to claim 1, and an adjusting tool configured to adjust a force component of the force applied to the rib elements by the coupling regions between parallel and antiparallel with respect to the longitudinal axis (z), wherein the adjusting tool is designed as a minimally invasive endoscopic instrument,

15. The system according to claim 14, wherein the adjusting tool is designed tweezer-like or pincer-like.

16. A method for implantation of an ossicular prosthesis (10; 20; 30; 40) according to claim 1, wherein the coupling regions (15; 25; 35; 45) within the head plate plane of the first fastening element have structures made of memory metal, and wherein the method comprises applying a force to the rib elements (14; 24; 34; 44) by heating the structures made of memory metal, with resulting deformation of the structures.

17. The method according to claim 16, wherein the heating takes place in a contactless manner by light irradiation.
